(19) United States
(12) Patent Application Publication
(10) Pub. No.: US 2011/0302829 A1
(43) Pub. Date: Dec. 15, 2011

(54) Fatty Acid Composition, Its Production and Use

(75) Inventors: Mark Brewer, Almere (NL); Torbjörn Kautto, Sandarne (SE); Matti Ravaska, Oulu (FI)

(73) Assignee: ARIZONA CHEMICAL, CE ALMERE (NL)

(21) Appl. No.: 13/218,397

(22) Filed: Aug. 25, 2011

Related U.S. Application Data
(63) Continuation of application No. 10/523,649, filed on Jul. 29, 2005.

(30) Foreign Application Priority Data
Aug. 5, 2002 (FI) 20021446
Dec. 3, 2002 (FI) 20022129

(51) Int. Cl. C10L 1/188; C07C 57/02

(52) U.S. Cl. 44/385; 554/224

(57) ABSTRACT

The present invention relates to a fatty acid composition which contains an effective amount of fatty acids providing improved low temperature stability of the composition. The present invention also relates to a process for producing a fatty acid composition wherein the process comprises the steps of selecting a crude tall oil having a fatty acid concentration and type capable of providing low temperature stability, distilling said crude tall oil to provide a fatty acid composition containing an effective amount of tall oil fatty acids providing low temperature stability. The fatty acid composition of the present invention is used as a fuel additive

The present invention relates to a fatty acid composition and to a process for production thereof, and to the use thereof. The fatty acid composition has excellent lubricity performance and good low temperature stability and it is used e.g. as a fuel additive in various types of fuel. Its low temperature properties make it useful also for other uses, such as ore flotation compositions and surfactant compositions. The present invention also relates to a fuel additive and a fuel having good low temperature properties. The preferred fatty acid composition is derived from tall oil.

### BACKGROUND

Crude tall oil is recovered from black liquors produced in the kraft pulping process. The composition of crude tall oil is not constant, it varies depending on factors such as cooking, washing, skimming, storage and most of all, the source of wood chips used in the pulping. The tall oil fatty acids are contained in the black liquor in the form of soaps. When the soaps are acidified free fatty acids and resin acids are obtained. The fatty acids and resin acids are normally separated in a separate processing stage by distillation.

Tall oil fatty acids are used in various applications, such as in fuel additive compositions due to their good lubricating properties. They are also used in ore flotation compositions and surfactant compositions.

Many vegetable and animal sources also provide fatty acids which can be used in similar applications.

Fuel additives are used in fuels in order to improve certain aspects of the fuel performance, e.g. lubricity, low temperature properties, or to reduce emissions from the engines. Fuel additives can also be used to allow the use of less polluting fuels in engines without engine damage.

The sulfur content of fuels has been lowered due to environmental legislation aimed at reducing air pollution from sulfur dioxide. The removal of sulfur compounds at the refinery has resulted in the lubricating properties of the fuel being reduced. This in turn has caused increased wear on engine components.

The wear problems in the engines have been solved by adding certain types of additives into the fuel to improve lubricity. Lubricity improvers may be of various types of chemistry including esters, fatty acids, amides and other nitrogen containing material, alcohols etc. Fatty acids have been found to be a cost effective solution. Fatty acid compositions from various sources, such as rapeseed, soya, sunflower, tall oil, have also been used as lubricating agents in fuels.

WO98/04656 discloses a fuel for diesel engines with a sulfur content less than 500 ppm. The fuel comprises at least 20 ppm of a lubricating additive formed by the combination of one monocarboxylic aliphatic hydrocarbon and at least one polycyclic hydrocarbon compound. The fuel contains additive more than 60 ppm when the combination is tall oil.

WO02/20703 discloses a fuel additive composition comprising the reaction product of a mixture of mixed fatty acid esters, a mono or di-(hydroxy alkyl amine) or mixtures thereof and a low temperature property enhancing effective amount of a low molecular weight ester, wherein the reaction mixture has a molar ratio of amine to total ester content in the range from 10.0 to 1.0.

U.S. Pat. No. 3,667,152 discloses a fuel composition comprising a mixture of hydrocarbons boiling in the range from 90 to 625° F. (32-329° C.), containing from 0.01 to 0.1 weight percent of tall oil fatty acid. The fatty acid was added in order to provide a fuel having high anti-wear, water separation and thermal stability properties.

WO01/38461 discloses the use of a flow improver for the prevention and/or inhibition of the crystallization of a fatty acid from a composition comprising the fatty acid.

During the wintertime insufficient low temperature stability of the fuel causes problems in cold climates. Anti-freezing agents and/or low temperature additives have been added to alleviate the problems. Despite the use it has been difficult to find fuel additives which improve lubricating properties and having a sufficient low temperature stability. The consequence of poor low temperature stability in use is the fuel additive separates from the fuel during storage. This may cause plugging resulting in blocking of filters and uneven dosing of the fuel. On the other hand a high amount of additives or many different types of additives in a fuel may cause problems as well.

Thus, there exists a need for fatty acids with improved low temperature properties.

It has now been found that certain fatty acid compositions provide desired low temperature properties. Moreover, it has been found that these fatty acid compositions can be found is tall oil fatty acids. These fatty acids can provide better low temperature properties than those that have been achieved so far in the fatty acid chemistry.

### SUMMARY OF THE INVENTION

The present invention relates to a fatty acid composition which fatty acid composition provides low temperature stability to the product that it is added to. The fatty acid composition is preferably used as a fuel additive since it also provides excellent lubricating properties and thus improves the over-all performance of the formulated fuel. As a lubricity improver the composition prevents wear on engines. A parameter which indicates the low temperature stability is the temperature at which the cloud point occurs. The fatty acid composition according to the present invention has a low cloud point. The low temperature properties of the fatty acid composition of the present invention may also be utilized in ore flotation techniques and surfactant compositions.

The preferred fatty acid composition of the present invention is a fatty acid composition having a specific distribution of fatty acids of different chain length and specific amounts of saturated and unsaturated fatty acids.

A fatty acid composition of the present invention contains an effective amount of fatty acids providing improved low temperature stability of the composition. The fatty acids are obtained from tall oil, vegetable fatty acids and/or animal fatty acids.

It has been found that unsaturated fatty acids improve the low temperature stability compared to saturated fatty acids. The low temperature stability of unsaturated fatty acids varies as well. Polyunsaturated fatty acids have better low temperature stability than monounsaturated fatty acids. The preferred fatty acids providing the low temperature stability are C18 fatty acids, preferably unsaturated C18 fatty acids, more preferably polyunsaturated C13 fatty acids. It has been found that especially C18;3 fatty acids, affect the low temperature properties in a beneficial way.

In a preferred embodiment the fatty acid composition contains fatty acids derived from plant sources such as tall oil and/or vegetable oils. A preferred composition contains less than 5%, preferably less than 3% saturated fatty acids calculated on the total weight of said fatty acids composition and more than 90%, preferably more than 95%, more preferably more than 98% unsaturated fatty acids calculated on the total weight of said fatty acids.

In the preferred embodiment of the invention the fatty acids providing the improved low temperature stability are derived from tall oil. Certain vegetable oils which are high in unsaturated and low in saturated fatty acids are also suitable for use in the invention. Such oils include linseed oil and oil from fish and/or seaweeds

In a preferred tall oil fatty acid composition of the present invention the content of the C18;3 fatty acids is more than 10%, preferably than 15%, more preferably more than 20%, most preferably more than 25% calculated on the total weight of said fatty acids. In an especially preferred embodiment the C18;3 fatty acid is at least predominantly pinolenic acid.

The amount of the C16 and C18 saturated fatty acids is preferably as low as possible. The preferred tall oil fatty acid composition of the present invention has the total content of C16;0, C17;0 and C18;0 fatty acids less than 2.2%, more preferably less than 1%, most preferably less than 0.5% calculated on the total weight of said fatty acids.

It is especially important that the content of C18;0 is very low i.e. that the composition contains very little or next to no stearic acid. The stearic acid level is preferably below 0.5%.

In a preferred tall oil fatty acid composition of the present invention the content of C20;0 fatty acids is less than 1%, preferably less than 0.5%.

The content of the resin acids in the fatty acid composition of the present invention is low. A tall oil fatty acid composition of the present invention has the content of the resin acids less than 10%, preferably less than 5%, more preferably less than 2%, most preferably less than 1%.

In a preferred tall oil fatty acid composition of the present invention the content of the C18;2 fatty acids is more than 30%, preferably than 40%, more preferably more than 50%.

In a preferred tall oil fatty acid composition of the present invention the content of the C18;1 fatty acids is less than 35%, preferably less than 25%, more preferably less than 20%.

Compositions having a high content of 18;3 and a very low content of C18;0 are preferred since they give very low cloud points. However, it has also been found that the whole composition plays a role in determining the cloud point. The following equation has been developed for use in determining whether any given fatty acid composition is likely to have the desired low temperature characteristics:

### DEFINITIONS

[C 16;0] means concentration of C16 saturated fatty acids
[C17;0] means concentration of C17 saturated fatty acids
[C18;0] means concentration of C18 saturated fatty acids
[C20;0] means concentration of C20 saturated fatty acids
[C18;1] means concentration of C18 mono-unsaturated fatty acids
[C18;2] means concentration of C18 di-unsaturated fatty acids
[C18;3] means concentration of C18 tri-unsaturated fatty acids
[Resin] means concentration of resin fatty acids

Concentrations are determined by standard ASTM GC method.

### Concentration Factors

### A=6.2

### B=1.32

### C=34.5

### D=0.075

### E=1.3

### F=−0.27

### G=−5.1

### H=17

Calculation of cloud point factor (Cp_{fac}) by equation I

Cp_{fac}=A·[C16;0]+B·[C17;0]+C·[C18;0]+D·[C20;0]+E·[C18;1]+F·[C18;2]+G·[C18;3]+H·[Resin]

If calculated according to the above mentioned equation, the composition has a low Cp_{fac}, i.e. a value below 0.4 the composition is likely to have low temperature properties. A Cp_{fac} value below 0.28 indicates a composition having a cloud point lower the −9° C., which is considered a very good value for low temperatures. Cloud point factors for typical standard prior art fatty acid compositions are in the order of 1.5 to 0.4.

The cloud point of the tall oil fatty acid composition according to the present invention is low and in a preferred embodiment it is lower than for fatty acid compositions previously known. The cloud point of the fatty acid composition according to the present invention is well below the freezing point of water. In order to be effective, it should be below −4° C. and preferably lower. In a preferred embodiment the cloud point is below −6° C., preferably below −9° C. With the composition of the present invention it is possible to provide a cloud point which is lower than −10° C., preferably lower than −15° C., more preferably lower than −20° C.

The present invention also relates to a process for producing a fatty acid composition comprising fatty acids. The process comprises the steps of selecting a crude tall oil having a fatty acid concentration and type, capable of providing low temperature stability for the process and distilling the crude tall oil to provide the desired fatty acid composition containing an effective amount of fatty acids to provide low temperature stability. The specific fatty acid distribution provides good low temperature properties to the composition.

Tall oil from softwood contains 35 to 55% fatty acids and 20 to 40% resin acids, while hardwood tall oil contains lower resin acid. The distribution of fatty acids within the tall oil varies with variations in pulp raw material. Thus, for instance trees grown in North America provide a different fatty acid distribution from trees grown in Scandinavia.

The fatty acid composition according to the present invention with improved low temperature performance is produced by the use of raw material selection. The crude tall oil which is used in the process of the present invention is selected based on its fatty acid concentration and type to provide a desired fatty acid composition. It is possible fairly accurately to predict the fatty acid distribution of the distilled tall oil fatty acid composition based on the initial fatty acid concentration and type of the crude tall oil since only minor changes occur during the distillation. Selection of crude tall oil may also include blending of different crude tall oils to provide a desired raw material. In a preferred process of the present invention the crude tall oil is derived from trees grown in a cold climate.

In a preferred crude tall oil for the process of the present invention more than 4% of the fatty acids are tripleunsaturated fatty acids calculated on the total weight of the fatty acids. A preferred crude tall oil has less than 1% of the fatty acids which are saturated fatty acids C18 or greater calculated on the total weight of the fatty acids. It is also preferred that the crude tall oil used has 0.3%, preferably less than 0.2%, more preferably less than 0.1% of the fatty acids of the crude tall oil are C18;0 fatty acids calculated on the total weight of the fatty acids.

The selected crude tall oil is then distilled using a conventional tall oil distilling procedure in order to obtain a fatty acid composition with a specific fatty acid distribution which provides improved low temperature properties.

It is also possible to provide a fatty acid composition of the present invention by blending the appropriate fatty acids derived from different sources.

The fatty acid composition according to the present invention can also be reacted into derivatives, such as esters, amides, and/or amine salts imidazolines, which also have improved low temperature properties. The esters can be mixtures of mono, di, tri esters of glycerol, and esters of pentaeryrithritol, trimethylolpropane, monoethylene glycol, neopentyl glycol and other polyalcohols, and methanol, ethanol, propanol, butanol and 2-Ethyl-hexanol and other mono alcohols and/or CnOHm, wherein n=1-30, m=1-6. The preferred derivatives are glycerol ester and diethanolamine derivatives.

The fatty acid composition of the present invention is used as a fuel additive as a lubricity improver. The fatty acid composition of the present invention may be added directly to the fuel or it may form a part of a fuel additive package, where such packages are common in the fuel additive industry. Other components that may be present in the fuel additive package are one or more of detergent, cold flow additive, antifoam, static dissipate, antioxidant and other additives used in the art.

The present invention also relates to a fuel additive which is stable at temperature below −4° C.

The present invention also relates to a fuel containing a fatty acid composition wherein the fuel contains an effective amount of a low temperature stable fatty acid composition lubricity enhancer of the present invention which is stable at temperature below −4° C. The fatty acid composition according to the present invention is used as a lubricating additive in fuels, such as diesel, gas oil, gasoline, aviation fuel and kerosene and mixtures. The fuel of the present invention can be of low sulfur content, i.e. less than 500 ppm, preferably less than 350 ppm, more preferably less than 50 ppm. The sulfur content of the fuel can be even as low as less than 15 ppm or less than 10 ppm. Typically, 10 to 1000 parts per million (ppm) of the fatty acid composition of the present invention in the fuel is necessary in order to afford improved lubricity to the fuel. It is also used in ore flotation compositions and surfactant compositions.

### DETAILED DESCRIPTION OF THE INVENTION

A fatty acid composition according to the present invention is preferably produced by distilling and fractioning crude tall oil to provide a controlled tall oil fatty acid composition. The crude tall oil is distilled using conventional distillation equipment and techniques. The fatty acid composition may also be obtained from vegetable or animal sources.

A fatty acid composition according to the present invention is also produced by blending fatty acids derived from different sources, such as tall oil, vegetable and animal fat. Preferable fatty acids are derived from rapeseed, soy, canola, linseed, Lung oil and fish oil. The fatty acids are derived from e.g. distillation fractions or refinery currents. The fatty acid composition of the present invention is made by blending high amount of C18;3 fatty acids and low amount saturated fatty acids with any other fatty acids.

The starting material for the process according to the present invention is selected based on the fatty acid concentration and type of the crude tall oil. By selecting a proper crude tall oil or a blend of tall oils the desired fatty acid composition is obtained. The properties of the fatty acid composition are determined by the amount of the different fatty acids types.

The appropriate crude tall oil is derived from e.g. sources which provide a specific fatty acid content, like trees grown in a special habitat. Different types of trees are grown in different habitat, this determines their characteristic fatty acid concentration and type. The selection of raw material for the crude tall oil affects the quality of the crude tall oil. For example crude tall oil derived from softwood has a different fatty acid composition than crude tall oil derived from hardwood, similarly tree varieties also influence the fatty acid composition.

The crude tall oil used in the present invention is preferably selected based on the tree type. It has been found trees grown in severe cold climate provide a preferred type of crude tall oil to provide a fatty acid composition with improved low temperature stability.

The distillation of the crude tall oil is performed in a conventional distillation system. The distillation system has preferably the steps of dewatering, depitching, heads removal, rosin removal, fatty acid removal and DTO (distilled tall oil) processing. Each of the steps can have single or multiple operations. The distillation process is performed either as a batch or a continuous process.

Each process step is performed using separation techniques which are known as such and which may include wiped, thin and/or falling film evaporators, fractional distillation using different types of column packing. Pressure and/or vacuum, temperature and residence time is also used in the separation techniques.

The crude tall oil is divided into heads, pitch, tall oil rosin, tall oil fatty acid and distilled tall oil. Water and light-volatile oil are distilled first from the crude tall oil. Water and light-volatiles are preferably further processed in other processes. The remaining crude tall oil is distilled further to provide a raw fatty acid distillate by removing high boiling pitch and rosin. The raw fatty acid distillate is fractioned into distilled tall oil and tall Oil fatty acids.

“Tall oil” as used in this description and the claims means extractives obtained from wood in kraft pulping. “Crude tall oil” is obtained by acidifying the tall oil soap recovered from the black liquor and it contains fatty acids, resin acids and neutral materials.

“Tall oil fatty acids” means the fatty acids obtained from the crude tall oil by distilling. Tall oil fatty acids in the present invention usually have a chain length C16 to C30. The percentages of the fatty acids in the present description and claims are calculated on the total weight of the fatty acid composition. The significant amount of fatty acid in the description and the claims is 0.1%. Any content of fatty acid other than the ones mentioned is insignificant in case the concentration is less than 0.1%.

The fatty acids are designated according to their carbon chain length and number of double bonds according to a standard nomenclature wherein e.g. C18;0 indicates a chain length of 18 carbon atoms and no double bonds while C20;4 indicates a chain length of 20 carbon atoms and 4 double bonds. The position of any double bonds is indicated by numbers e.g. as 18;2-9,12 wherein 9 and 12 indicates the positions of the two double bonds.

“Resin acids” are monocarboxylic diterpene acids, the most common of which has the molecular formula C₂₀H₃₀₀O₂. The resin-based acids can be selected from abietic acid, dihydroabietic acid, dehydroabietic acid, neoabietic acid, pimaric acid, levopimaric acid, palustric acid, isopimaric and other derivatives based on the diterpene structure.

“Unsaponifiables” are neutral substances found in the tall oil which include higher fatty alcohols, esters, plant sterols and some hydrocarbons.

“Low temperature stability” as used in this description and the claims means that the fatty acid composition has a low cloud point. Cloud point is defined as a temperature of a liquid specimen, when a wax crystal structure that is similar in appearance to a cloud is formed upon cooling under prescribed conditions.

The content of the unsaturated and saturated fatty acids is controlled in order to achieve the desired low temperature properties. Especially the content of polyunsaturated fatty acids affects the stability. Therefore, the aim of the invention is to have as much unsaturated fatty acids as possible and only minor amount of saturated fatty acids in the fatty acid composition according to the present invention.

In the present invention it has been found that some crude tall oil contain an exceptionally high level of polyunsaturated C18 fatty acids. When these crude tall oils are distilled with conventional techniques a fatty acid composition is obtained which contains a significant amount of said polyunsaturated C18 fatty acids. The composition thus obtained has a surprisingly effective influence on the low temperature stability of the composition.

It has been found that the content of C18,3 fatty acids is the most critical for the low temperature stability of a fatty acid composition. Therefore the content of C18;3 fatty acids is as high as possible in the composition. An especially preferred C18;3 fatty acid is pinolenic acid since it enhances low temperature stability very effectively. The content C18;2 fatty acids is preferably also high in the composition, since it also influences the stability positively.

Saturated fatty acids have a negative influence on the low temperature stability of a fatty acid composition. Especially the contents of C18;0, C17;0 and C16;0 fatty acids must be low in the fatty acid composition of the present invention in order to have good low temperature stability. Also the content of C20;0 fatty acids is preferably low.

The fatty acid composition of the present invention remains stable well below the freezing point of water. The compositions are stable at temperatures below −4° C., preferably below −6° C., more preferably −10° C. This makes the fatty acid composition effective also at temperatures lower than corresponding prior art products. It is even possible to obtain a fatty acid composition which is stable at temperatures below −15° C. or even below −20° C.

Low temperature performance of a fatty acid composition is screened using e.g. cloud point, DSC, long term storage at different temperatures (e.g. 5, 0, −15° C.) and cold filtering plugging point (CFPP).

The cloud point may be tested e.g. with an automatic ASTM standard method D5771. The cloud point is defined as a temperature of a liquid specimen, when a wax crystal structure that is similar in appearance to a cloud is formed upon cooling under prescribed conditions. The test is performed by inserting a sample into an apparatus and the sample is cooled according to a cooling profile. The sample is continuously monitored by an optical system for the formation of a crystalline structure. When the crystallization of the wax in the specimen is detected, the temperature is recorded.

Low temperature stability may also be determined by monitoring the appearance of a cooled sample over an extended period of time. A sample is placed in a container which is placed into a cooled environment. The clarity of the sample is visually examined and judged on a predetermined scale on a periodic basis, e.g. daily or weekly.

Differential scanning calorimetry (DSC) is also used to determine the low temperature stability. A sample is subjected to a heating and cooling regime: heat from 25° C. to 100° C. at the rate of 50° C./min, hold at 100° C. for 2 min, cool from 100° C. to −50° C. at the rate of 10° C./min, hold at −50° C. for 2 min and heat from −50° C. to 100° C. at 20° C./min. The exotherms and the endotherms during the heating and cooling are measured with DSC. The sample which has a relatively lower crystallization temperature has a better low temperature stability.

The quality of fuel additives is determined by e.g. the test method IP 450 by Institute of Petroleum. The test method assesses the lubricating property of diesel fuels and fuels which may contain lubricity enhancing additives using a high-frequency reciprocating rig (HFRR). Lubricity fuel additive performance requirements e.g. for Europe is HFRR <460 μm WSD (wear scar diameter).

The fatty and resin acids in a tall oil fractionation product are determined using capillary gas chromatography according to a ASTM standard method D 5974. The amount of the individual fatty acids and resin acids are determined using capillary gas chromatography separation of the volatile methyl esters of these acids.

The fatty acid composition of the present invention may be used as a fuel additive as such or it may be blended with other additives before being added to the fuel. The fatty acid composition is effective as a lubricity improver. The amount of additives added to the fuel may vary depending on many factors such as the type of the fuel and the sulfur content of the fuel. The sulfur content of the fuel is preferably less than 500 ppm, more preferably less than 350 ppm, most preferably less than 50 ppm. The sulfur content of the fuel can be even as low as less than 15 ppm or less than 10 ppm.

Typically, 10 to 1000 parts per million (ppm) of the fatty acid composition of the present invention in the fuel is necessary in order to afford improved lubricity to the fuel.

In the ore recovery processes the fatty acid composition may be substituted for the conventional fatty acid composition used in the froth flotation. This is especially useful in cold climates where the flotation in the wintertime has had problems with the poor stability of conventional compositions at low temperatures such as below −4° C. and especially below −9° C.

In the following the present invention will be illustrated by some examples which describe some embodiments of the invention.

### Example 1

Crude tall oil was selected and analyzed by the ASTM standard method D 5974. The contents of different fatty acids were

| | |
|---|---|
| C 16:0 | 0.8 |
| C 17:0 | 0.4 |
| C 18:0 | 0.2 |
| C 18:1 | 9.3 |
| C 18:2 | 15.2 |
| C 18:3 | 5.4 |
| C 20:0 | 0.4 |
| resin acids | 34 |

The crude tall oil was distilled in accordance with the present invention to provide a fatty acid composition. The fatty acid composition obtained was analyzed and the content of the fatty acids was:

| | |
|---|---|
| C 16:0 | 0.1 |
| C 17:0 | 0.2 |
| C 18:0 | 0.7 |
| C 18:1 | 29.7 |
| C 18:2 | 45 |
| C 18:3 | 14 |
| C 20:0 | 0.3 |
| resin acids | <2 |

The cloud point of the fatty acid composition was analyzed with the ASTM standard method D5771. The cloud point was −1.1° C. The composition was also analyzed by DSC and the first thermal event on second cooling was −21° C. The cloud point factor calculated by equation I was 0.14.

The lubricity performance of the fatty acid composition was good and the composition was useful for being added to a fuel as a low temperature stable additive.

### Example 2

The crude tall oil of Example 1 was distilled also in a second trial.

The fatty acid composition obtained was analyzed and the content of the fatty acids was:

| | |
|---|---|
| C 16:0 | 0.2 |
| C 17:0 | 0.2 |
| C 18:0 | 0.8 |
| C 18:1 | 30.2 |
| C 18:2 | 47.8 |
| C 18:3 | 12.6 |
| C 20:0 | 1.0 |
| resin acids | <2 |

The cloud point of the fatty acid composition was analyzed with the ASTM standard method D5771. The cloud point of the fatty acid composition was −15° C. and it was useful as a low temperature stable additive. The cloud point factor calculated according to equation I was 0.25.

### Example 3

### Reference Example

Crude tall oil from American sources was fractionated into a tall oil fatty acid composition with a resin acid content of about 2%.

The fatty acid composition obtained was analyzed and the fatty acids comprised:

| | |
|---|---|
| C 16:0 | 1 |
| C 17:0 | 0.3 |
| C 18:0 | 2.3 |
| C 18:1 | 48.4 |
| C 18:2 | 38 |
| C 18:3 | 4.8 |
| C 20:0 | 0.1 |
| resin acids | <2 |

The cloud point of the fatty acid composition obtained from the American crude tall oil was 7° C. The content of the C18;3 fatty acid content in oil fatty acid composition was only (4.8%). The cloud point factor of the composition was found to be 1.48.

### Example 4

Two fatty acid compositions were tested for their low temperature stability. The first one was the American fatty acid composition of Example 3.

The second fatty acid composition was obtained from trees grown in a cold climate region. It contained the following amounts of fatty acids

| | |
|---|---|
| C 16:0 | 0.5 |
| C 17:0 | 0.2 |
| C 18:0 | 0.5 |
| C 18:1 | 24.3 |
| C 18:2 | 48.9 |
| C 18:3 | 19.5 |
| C 20:0 | 0.5 |
| resin acids | <2 |

The cloud point of the cold zone tall oil fatty acid composition was −18° C., whereas the cloud point of the American tall oil fatty acid composition was 7° C. DSC analysis of the cold zone composition indicated a first thermal event on second cooling at −28° C. The cloud point factor was found to be −0.52.

It should be noted that the fatty acid compositions of example 2 and 4 which had excellent low temperature stability (cloud point −12° C. and −18° C., respectively) also showed significantly improved lubricity properties.

### Example 5

The fatty acid compositions obtained in examples 1, 2 and 4 were tested for their lubricity performance by HFRR (IP450) in <500 ppm S Diesel fuel. Wear scar diameters in this test are acceptable when they are less than 460 μm. The results are shown below.

| | Example | | |
|---|---|---|---|
| Treat rate (mg/l) | 1 | 4 | 2 |
| Base fuel | 594 | 590 | 590 |
| 50 | 476 | 367 | 350 |
| 100 | 401 | 375 | 375 |
| 200 | 377 | 363 | 374 |

All of the tested tall oil fatty acid compositions were acceptable from a lubricity point of view.

### Example 6

A crude tall oil obtained from softwood was distilled and was found to provide a fatty acid composition as follows:

| | |
|---|---|
| C 16:0 | 0.9 |
| C 17:0 | 0.2 |
| C 18:0 | 0.3 |
| C 20:0 | 0.1 |
| C 18:1 | 31.1 |
| C 18:2 | 38.2 |
| C 18:3 | 10.2 |
| resin acids | 0.5 |

The composition had a cloud point of −15.0° and an acid value of 195.0. The composition was suitable as for cold climate fuels. Its cloud point factor was 0.03.

The present invention has been illustrated in detail by the above examples. It is evident to those skilled in the art that the invention may be used in many different ways and many different applications.

1. A fatty acid composition characterized in that said composition contains less than 3% saturated fatty acids, more than 10% C18;3 fatty acids, more than 30% C18;2 fatty acids and less than 35% C18;1 fatty acids, said fatty acids providing improved low temperature stability of the composition, and that the cloud point of said fatty acid composition is lower than −4° C., and said composition lacks a paraffine dispergent.

2. A fatty acid composition according to claim 1 characterized in that said fatty acids are derived from plant sources.

3. A fatty acid composition according to claim 1 or 2 characterized in that said fatty acids are derived from tall oil or vegetable sources.

4. A fatty acid composition according to claim 1, 2 or 3 characterized in that the composition contains less than 1.5% saturated fatty acids and more than 90%, preferably more than 95%, more preferably more than 98% unsaturated fatty acids.

5. A fatty acid composition according to claim 4 characterized in that the content of the C18;3 fatty acids is more than 15%, preferably more than 20%, more preferably more than 25%.

6. A fatty acid composition according to claim 5 characterized in that said C18;3 fatty acid is pinolenic acid.

7. A fatty acid composition according to claim 4 or 5 characterized in that the total content of C16;0, C17;0 and C18;0 fatty acids is less than 2.2%, more preferably less than 1%, most preferably less than 0.5%,

8. A fatty acid composition according to claim 4 characterized in that the content of C20;0 fatty acids is less than 1%, preferably less than 0.5%.

9. A fatty acid composition according to claim 4 characterized in that the content of the resin acids is less than 5%, preferably less than 2%, more preferably less than 1%.

10. A fatty acid composition according to claim 4 characterized in that the content of the C18;2 fatty acids is more than 40%, preferably more than 50%.

11. A fatty acid composition according to claim 4 characterized in that the content of the C18;1 fatty acids is less than 25%, preferably less than 20%.

12. A fatty acid composition according to any one of the preceding claims characterized in that the composition contains more than 10%, preferably more than 15% C18;3 fatty acids and more than 30%, preferably more than 40% C18;2 fatty acids and less than 1%; preferably less than 0.5% C18;0 fatty acids and less than 2%, preferably less than 1% resin acids and the total of saturated fatty acids is less than 1.5%.

13. A fatty acid composition according to any one of the preceding claims having a cloud point factor below 0.28 calculated according to the equation I Cp_{fac}=A·[C16;0]+B·[C17;0]+C·[C18;0]+D·[C20;0]+E·[C18;1]+F·[C18;2]+G·[C18;3]+H·[Resin], wherein
[C16;0] means concentration of C16 saturated fatty acids, [C17;0] means concentration of C17 saturated fatty acids, [C18;0] means concentration of C18 saturated fatty acids, [C20;0] means concentration of C20 saturated fatty acids, [C18;1] means concentration of C18 mono-unsaturated fatty acids, [C18;2] means concentration of C18 di-unsaturated fatty acids, [C18;3] means concentration of C18 tri-unsaturated fatty acids, [Resin] means concentration of C16 resin fatty acids and concentration factors are A=6.2, B=1.32, C=34.5, D=0.075, E=1.3, F=−0.27, G=−5.1 and H=17.

14. A fatty acid composition according to any one of the preceding claims characterized in that the cloud point of said fatty acid composition is lower than −6° C., preferably lower than −10° C., more preferably lower than −15° C., most preferably lower than −20° C.

15. An ester characterized in that said ester is produced from fatty acid composition according to claim 1.

16. A glycerol ester characterized in that said glycerol ester is produced from fatty acid composition according to claim 1.

17. A process for producing a fatty acid composition according to claim 1 characterized in that said process comprises the steps of
selecting a crude tall oil having a fatty acid concentration and type capable of providing low temperature stability
distilling said crude tall oil to provide a fatty acid composition containing an effective amount of tall oil fatty acids providing low temperature stability.

18. A process according to claim 17 characterized in that selecting includes blending of different crude tall oils.

19. A process according to claim 17 characterized in that said crude tall oil is derived from trees grown in a cold climate.

20. A process according to claim 17 characterized in that more than 4% of the fatty acids of the crude tall oil are triple unsaturated fatty acids.

21. A process according to claim 17 characterized in that less than 1% of the fatty acids of the crude tall oil are saturated fatty acids of C18 or greater.

22. A process according to claim 13 characterized in that less than 0.3%, preferably less than 0.2%, more preferably less than 0.1% of the fatty acids of the crude tall oil are C18;0 fatty acids.

23. Use of a fatty acid composition according to claim 1 as a fuel additive.

24. Use of a fatty acid composition according to claim 1 as a lubricity improver in fuel.

25. Use according to claim 24 characterized in that said lubricity improver forms a part of a fuel additive package containing other additives.

26. Use according to claim 25 characterized in that said other additives are one or more of detergent, cold flow additive, antifoam, static dissipate and/or antioxidant.

27. Use of an ester according to claim 15 or 16 as a fuel additive.

28. A fuel additive comprising fatty acid composition according to claim 1 characterized in that it is stable at temperature below −4° C.

29. A fuel containing a fatty acid additive characterized in that said fuel contains an effective amount of a low temperature stable fatty acid lubricity enhancer according to claim 1 which is stable at temperature below −4° C.

30. A fuel according to claim 29 characterized in that said fuel is diesel, gas oil, gasoline, aviation fuel or kerosene, or a mixture thereof.

31. A fuel according to claim 29 characterized in that sulfur content of said fuel is less than 500 ppm, preferably less than 350 ppm, more preferably less than 50 ppm, more preferably less than 15 ppm, most preferably less than 10 ppm.

32. A fuel according to claim 29 characterized in that said fuel contains 10 to 1000 ppm of said fatty acid lubricity enhancer.
